(19) United States
(12) Patent Application Publication
(10) Pub. No.: US 2020/0188253 A1
(43) Pub. Date: Jun. 18, 2020

(54) TWO-COMPONENT HAIR CARE AGENT WITH COLOR INDICATOR FOR THE IDENTIFICATION OF THE OPTIMUM PH VALUE, METHOD OF APPLYING THE SAME AND USING THE SAME

(71) Applicant: Henkel AG & Co. KGaA, Duesseldorf (DE)

(72) Inventors: RENE KROHN, Norderstedt (DE); ERIK SCHULZE ZUR WIESCHE, Hamburg (DE)

(73) Assignee: Henkel AG & Co. KGaA, Duesseldorf (DE)

(21) Appl. No.: 16/715,223

(22) Filed: Dec. 16, 2019

(30) Foreign Application Priority Data
Dec. 18, 2018 (DE) 10 2018 222 132.4

(51) Int. Cl. A61K 8/34; A61K 8/891; A61K 8/46; A61K 8/41; A61K 8/36; A61K 8/37; A61K 8/31; A61K 8/73; A61K 8/06; A61Q 5/12

(52) U.S. Cl. CPC A61K 8/342; A61K 2800/30; A61K 8/891; A61K 8/463; A61K 8/41; A61K 8/347; A61K 8/36; A61K 8/37; A61K 8/315; A61K 8/731; A61K 8/062; A61Q 5/12; A61K 2800/45; A61K 2800/882; A61K 8/345

(57) ABSTRACT

The present disclosure provides a two-component hair care agent including a first component and a second component. The first component and the second component are provided separately from one another. The first component or the second component has a color indicator showing a color change in a pH-value range of from about 3 to about 6, the first component has a pH value in a range of from about 2 to about 4and the second component has a pH value in a range of greater than about 4 to about 9. The present disclosure also provides a method for applying the two-component hair care agent and a method of using the two-component hair care agent.

### CROSS-REFERENCE TO RELATED APPLICATION

This application claims priority to German Patent Application No. 10 2018 222 132.4, filed Dec. 18, 2018, which is incorporated herein by reference in its entirety.

### TECHNICAL FIELD

The present disclosure relates to the field of production of hair care agents in cosmetics. In particular, the present disclosure relates to a two-component hair care agent comprising two components having different ingredients provided separately from one another.

### BACKGROUND

Cosmetic compositions which, apart from, for example, some colorants, usually include only one ready-to-use mixed component, have long been known for the cleaning and/or care of the hair and are regularly improved or adapted to the changing needs of consumers. For example, consumers expect modern hair conditioning agents to clean, smooth, comb easily, soften, gloss, and render hair easy to style, moisturize, protect against dandruff and give hair more volume. There is a variety of active ingredients known which can fulfill one or more of the aforementioned requirements in one such cosmetic agent. However, the presence of a large amount of different active ingredients can destabilize a hair cosmetic, which is why there is usually an attempt to fulfill as many of the aforementioned requirements as possible using a small number of well-tolerated active ingredients.

A wide variety of different components can also limit formulation freedom in the development of cosmetic agents because components interact with one another or interfere with the structure. For example, substances can interfere with the desired structure of an emulsion, wherein the desired structure of the emulsion is directly associated with a certain advantageous cosmetic effect. The formulation freedom is further limited by the requirement for good biodegradability, regulatory limitations, etc. In this respect, it is highly desirable to provide additional degrees of freedom for cosmetic formulations.

The fact that cosmetics appeal to their users on an emotional level is also not an insignificant aspect. Cosmetic products whose use is pleasurable, interesting or otherwise associated with positive emotional experiences will, in simple terms, typically be preferred to boring products, even when the achievable end result is equivalent to a cosmetic application.

### SUMMARY

The present disclosure described herein has been achieved in view of the above-mentioned requirements or desirable properties and the underlying object is firstly to provide a cosmetic agent, in particular a hair care or conditioning agent, which constitutes an emulsion that can be easily produced. In particular, the agent should provide the possibility of offering maximum formulation freedom. On the other hand, it is also the object underlying the present disclosure to provide a cosmetic agent, in particular a hair care or hair conditioning agent, which offers a certain sense of achievement in the preparation and application.

The problem is solved by a first aspect of the present disclosure. This relates to a two-component hair care agent comprising a first component and a second component, wherein the first component and the second component are provided separately from one another, wherein the first component or the second component has a color indicator showing a color change in a pH-value range of from about 3 to about 6, preferably from about 3.5 to about 5.5, particularly preferably from about 4 to about 5, wherein the first component has a pH value in a range of from about 2 to about 4, preferably from about 2.3 to about 3.7, particularly preferably from about 2.5 to about 3.5, and wherein the second component having a pH value in a range of greater than from about 4 to about 9, preferably from about 4.5 to about 8.5, particularly preferably from about 5 to about 8.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

On the one hand, the degrees of freedom in the formulation of a cosmetic agent considerably increases, for example, by ingredients that cannot normally be used together, because this would impair storage stability, yet can be used in a product in which they are spatially separated until just before use as constituents of different components, or in which different conditions can be created in the various components, for example, with regard to the pH value or other relevant parameters. This freedom to make the pH value of the components different is actually used in the present disclosure. In this way, various ingredients in the component can be stored with the pH value which is more favorable for their stability, and in addition, a mixing of the components can achieve a pH value more favorable for hair care in the resulting mixture. The storage stability of certain ingredients is significantly increased by this approach. Hydrolysis-sensitive substances in a basic component can thus be provided in acid. The use of many substances in storable cosmetic products is made possible in the first place.

It should be noted at this point that anhydrous components can also be used as contemplated herein. When this is the case, and a pH value of such a component is given, the pH value indicated is the pH which results from thoroughly and uniformly mixing the component with water in a about 1:1 volume ratio.

In addition, it should be noted at this point that “anhydrous” in the context of the present disclosure means that from 0 to about 5% by weight of water, preferably from 0 to about 3% by weight, particularly preferably from 0 to about 1% by weight, most preferably from 0 to about 0.5% by weight of water is present, in each case based on the total weight of the designated “anhydrous” component.

Apart from that already mentioned, the first aspect provides a vivid sense of achievement in the preparation of the cosmetic product for use since the color indicator contained is so chosen to achieve, through an occurring color change, the achievement of a provided desirable mixing ratio and to indicate the uniformity of the mixing by the uniformity of the color change. This gives the user a visual confirmation that the product has been optimally mixed and is to be seen as a positive “do-it-yourself” success experience.

In the two-component hair care agent as contemplated herein, the color indicator, depending on its color change range, is preferably used in one of the first component and the second component, such that a desired mixing ratio of the first component and the second component, while maintaining and uniformly mixing the first component and the second component, sets a pH value in the resulting mixture in the range from about 3 to about 6, preferably from about 3.5 to about 5.5, particularly preferably from about 4 to about 5, indicated by the color change. Maintaining these pH value ranges results in the best care effect on the hair. Color indicators which can be used as contemplated herein are, for example, bromothymol blue or methyl red. Such selection of the indicator and use in the respectively suitable phase ensures that the attainment of the desirable pH value in the ready-to-use mixture and also that a good mixing of the same are safe and reproducible.

Apart from that already mentioned, in the two-component hair care agent as contemplated herein, the first component and the second component are each, independently of one another, preferably a hydrophilic phase, a lipophilic phase or an emulsion. The advantage of this free selectability is that there is a particularly high degree of freedom in the selection of ingredients and the product can be given the respective desirable properties in a wide range.

Apart from that already mentioned, in the two-component hair care agent as contemplated herein, the first component and the second component are preferably liquid at a temperature of about 20° C. and a pressure of about 10⁵ Pa. The advantage is that at normal housing temperature and pressure conditions, both components are in liquid form and can be mixed well and then applied.

Apart from that already mentioned, it is preferred in the two-component hair care agent as contemplated herein that it contains no oxidizing agent, no substantive dye and no oxidation dye precursor for dyeing keratinic fibers, in particular human hair. One advantage of this is the ease of use, without having to take necessary measures to prevent staining of skin, clothing or bathroom utensils with hair coloring agents.

Apart from that already mentioned, it is preferred in the two-component hair care agent as contemplated herein that the color indicator is methyl orange, methyl red, resorcinol yellow, quinaldine red, Congo red, bromocresol green or bromothymol blue. Such selection of the indicator as contemplated herein and use in the respectively suitable phase ensures that the attainment of the desirable pH value in the ready-to-use mixture and also that a good mixing of the same are safe and reproducible.

Apart from that already mentioned, it is preferred in the two-component hair care agent as contemplated herein that the color indicator is present in this first or second component in a concentration of from about 0.000001% by weight to about 0.1% by weight, preferably from about 0.0001% by weight to about 0.05% by weight, more preferably from about 0.0005% by weight to about 0.01% by weight, based on the total component containing the color indicator. The advantage is that in these concentration ranges, a clearly visible color, a clearly visible color change and a good visibility of the mixing quality can be achieved without reaching a disturbingly high concentration of the color indicator.

Apart from that already mentioned, it is preferred in the two-component hair care agent of the present disclosure that the first component and the second component are provided in a weight ratio of from about 1:10 to about 10:1, preferably from about 1:5 to about 5:1, particularly preferably from about 1:2 to about 2:1, most preferably about 1:1. The advantage is that no excessively small amounts that are difficult to dose have to be dosed.

Further aspects of the present disclosure are described in the following. Unless there is a specific obstacle, features previously described for the products may also be found in these further aspects. Apart from that already mentioned, the object underlying the present disclosure is solved by a second aspect, a method for applying the two-component hair care agent according to the first aspect, comprising a step of mixing the first component and the second component, so that a mixture of the first component and the second component is obtained. The advantage is that through such an experience, hair care formulations previously not commercially usable are obtained, the conventional hair care products can achieve superior cleaning and/or care performance.

A preferred development of this aspect is a method for applying the two-component hair care agent further comprising, in addition to mixing as described above, a step of applying the mixture of the first component and the second component to keratinic fibers, in particular to human hair, and optionally exposing to water and/or rinsing with water. The advantage is that the hairs treated with the products as contemplated herein can benefit from a superior care performance and have to a particularly great extent desirable properties such as softness, brilliance and absence of frizz.

Apart from that already mentioned, there is a third aspect of the present disclosure, the use of the two-component hair care composition according to the first aspect for the care, preferably for the conditioning, of keratinic fibers, in particular of human hair. The advantage is that the hairs treated with the products as contemplated herein can benefit from a superior care performance and have to a particularly great extent desirable properties such as softness, brilliance and absence of frizz.

Concrete embodiments of the present disclosure are the following formulations. All specifications are in terms of percent by weight based on the total weight of a component and “ad 100” means that with the corresponding substance, the missing percentages by weight are supplemented to 100 percent by weight. Formulation 1

### Component 1 Basic

| Isododecane | ad 100 |
|---|---|
| Cetearyl alcohol | 9.00 |
| Myristyl alcohol | 15.00 |
| Caprylic/Capric Triglyceride (INCI) | 5.00 |
| Dimethicone 5 cst (INCI) | 30.00 |
| Bromothymol blue | 0.005 |
| Monoethanolamine | 0.010 |
| Perfume | 1.40 |

### Component 2 Acidic

| Water | ad 100 |
|---|---|
| Sodium benzoate | 0.50 |
| Lactic acid | 0.90 |
| Stearamidopropyl Dimethylamine (INCI) | 0.80 |
| Cetrimonium Chloride (INCI) | 1.50 |

### Formulation 2

### Component 1 Acidic

| Water | ad 100 |
|---|---|
| Isopropyl myristate | 2.00 |
| Cetearyl alcohol | 6.00 |
| Cetylpalmitate | 1.00 |
| Behentrimonium chloride | 2.00 |
| Cetrimonium chloride | 1.50 |
| Distearoylethyl Hydroxyethylmonium Methosulfate | 1.50 |
| (INCI) | |
| Guar Hydroxypropyltrimonium Chloride (INCI) | 0.20 |
| Stearamidopropyl Dimethylamine (INCI) | 0.80 |
| Methylparaben | 0.30 |
| Phenoxyethanol | 0.40 |
| Lactic acid | 1.00 |
| Perfume | 1.40 |

### Component 2 Basic

| Water | ad 100 |
|---|---|
| Propylene glycol | 23.00 |
| Hydroxyethyl cellulose | 1.00 |
| Behenoyl PG-Trimonium Chloride (INCI) | 2.00 |
| Laureth-4 (INCI) | 1.00 |
| Dimethicone (INCI), Dimethiconol (INCI) | 20.00 |
| Methylparaben | 0.30 |
| Phenoxyethanol | 0.40 |
| Bromothymol blue | 0.005 |
| Monoethanolamine | 0.150 |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. A two-component hair care agent, comprising:
a first component; and
a second component,
wherein the first component and the second component are provided separately from one another,
wherein the first component or the second component has a color indicator showing a color change in a pH-value range of from about 3 to about 6,
wherein the first component has a pH value in a range of from about 2 to about 4, and
wherein the second component has a pH value in a range of greater than about 4 to about 9.

2. The two-component hair care agent according claim 1, wherein the color indicator, depending on a color change range of the color indicator, such that a desired mixing ratio of the first component and the second component while maintaining and uniformly mixing the first component and the second component, sets a pH value in the resulting mixture of the first and second components in a range of from about 3 to about 6 indicated by the color change.

3. The two-component hair care agent according to claim 1, wherein the first component and the second component are each independently of one another a hydrophilic phase, a lipophilic phase, or an emulsion.

4. The two-component hair care composition according to claim 1, wherein the first component and the second component are liquid at a temperature of about 20° C. and a pressure of about 10⁵ Pa.

5. The two-component hair care composition according to claim 1, which comprises no oxidizing agent, no substantive dye, and no oxidation dye precursor for dyeing keratinic fibers.

6. The two-component hair care composition according to claim 1, wherein the color indicator is methyl orange, methyl red, resorcinol yellow, quinaldine red, Congo red, bromocresol green, or bromothymol blue.

7. The two-component hair care agent according to claim 1, wherein the color indicator is present in the first component or the second component in a concentration of from about 0.000001% by weight to about 0.1% by weight based on the total component containing the color indicator.

8. The two-component hair care agent according to claim 7, wherein the color indicator is present in the first component or the second component in a concentration of from about 0.0005% by weight to about 0.01% by weight, based on the total component containing the color indicator.

9. The two-component hair care agent according to claim 1, wherein the first component and the second component are provided in a weight ratio of from about 1:10 to about 10:1.

10. The two-component hair care agent according to claim 9, wherein the first component and the second component are provided in a weight ratio of from about 1:2 to about 2:1.

11. The two-component hair care agent according to claim 1, wherein the first component or the second component has the color indicator showing the color change in the pH-value range of from about 3.5 to about 5.5.

12. The two-component hair care agent according to claim 1, wherein the first component has a pH value in the range of from about 2.5 to about 3.5.

13. The two-component hair care agent according to claim 1, wherein the second component has a pH value in the range of from about 5 to about 8.

14. A method of using the two-component hair care agent of claim 1, the method comprising the step of mixing the first component and the second component to obtain a mixture of the first component and the second component.

15. The method of using the two-component hair care agent according to claim 14, further comprising the step of applying the mixture of the first component and the second component to keratinic fibers, and optionally exposing the mixture to water and/or rinsing the mixture with water.

16. A two-component hair care agent, comprising:
a first component having a pH value in a range of from about 2.5 to about 3.5; and
a second component having a pH value is a range of from about 5 to about 8,
wherein the first and second components are provided separately from one another,
wherein the first component and the second component are provided in a weight ratio of from about 1:2 to about 2:1, and
wherein the first component or the second component has a color indicator showing a color change in a pH-value range of from about 4 to about 5.

17. The two-component hair care agent of claim 16, wherein the first component comprises:
about 9% by weight of cetearyl alcohol;
about 15% by weight of myristyl alcohol;
about 5% by weight Caprylic/Capric Triglyceride (INCI);
about 30% by weight of Dimethicone 5cst (INCI);
about 0.005% by weight bromothymol blue;
about 0.01% by weight of monoethanolamine;
about 1.4% by weight of a perfume; and
the balance being isododecane,
wherein all of the weight percentages are based on a total weight of the first component; and
wherein the second component comprises:
about 0.5% by weight sodium benzoate;
about 0.9% by weight lactic acid;
about 0.8% by weight Stearamidopropyl Dimethylamine (INCI);
about 1.5% by weight Cetrimonium Chloride (INCI); and
the balance being water,
wherein all of the weight percentages are based on a total weight of the second component.

18. The two-component hair care agent according to claim 16, wherein the first component comprises:
about 2% by weight of isopropyl myristate;
about 6% by weight of cetearyl alcohol,
about 1% by weight cetylpalmitate,
about 2% by weight behentrimonium chloride,
about 1.5% by weight cetrimonium chloride,
about 1.5% by weight Distearoylethyl Hydroxyethylmonium Methosulfate (INCI),
about 0.2% by weight Guar Hydroxypropyltrimonium Chloride (INCI),
about 0.8% by weight Stearamidopropyl Dimethylamine (INCI),
about 0.3% methylparaben,
about 0.4% by weight phenoxyethanol,
about 1% by weight lactic acid,
about 1.4% by weight perfume, and
the balance being water,
wherein all of the weight percentages are based on a total weight of the first component; and
wherein the second component comprises:
about 23% by weight propylene glycol;
about 1% by weight hydroxyethyl cellulose;
about 2% by weight Behenoyl PG-Trimonium Chloride;
about 1% by weight Laureth-4;
about 20% by weight of Dimethicone (INCI);
about 0.3% by weight methylparaben;
about 0.4% by weight phenoxyethanol;
about 0.005% by weight bromothymol blue;
about 0.15% by weight monoethanolamine; and
the balance being water,
wherein all of the weight percentages are based on a total weight of the second component.
